(19) United States
(12) Patent Application Publication
(10) Pub. No.: US 2007/0066901 A1
(43) Pub. Date: Mar. 22, 2007

(54) Fluid driven mechanical scanning with an ultrsound transducer array

(75) Inventors: Sean T. Hansen, Palo Alto, CA (US); John P. Mohr III, Aptos, CA (US); John D. Marshall, Los Gatos, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/233,642

(22) Filed: Sep. 22, 2005

(51) Int. Cl. A61B 8/14

(52) U.S. Cl. 600/459

(57) ABSTRACT

A wobbler drive mechanism is provided for mechanically scanning an ultrasound transducer array. A fluid drive moves the transducer array. A pump causes fluid flow. The fluid flow transfers energy to the transducer array for moving the transducer array.

### BACKGROUND

The present embodiments relate to mechanically scanned ultrasound transducers. In particular, drive mechanisms for mechanically scanned ultrasound transducers are provided.

Ultrasonic transducers are wobbled with motors. The motors mechanically scan the transducer from side-to-side. The motor is located close to the ultrasound transducer. For hand-held probes, wobbler arrays are provided for three-dimensional imaging. For small probes, such as catheters, the motor is located in the tip of the probe catheter or outside the patient in a control box. If the motor is located outside the catheter, a drive and cable transfers the motor's rotational motion to the ultrasound transducer for mechanical scanning.

Electric motors emit electromagnetic interference (EMI), which can corrupt the received ultrasound signals from the transducer, particularly if the signals from the transducer are not amplified at the transducer. Effective shielding may be difficult in space-constrained applications such as catheter ultrasound imaging. In addition, miniature electric motors may be prohibitively expensive to include in a disposable catheter ultrasonic probe. By locating the motor away from the transducer, transferring the rotational energy of the motor is challenging since the catheter diameter is narrow and most of the cross-sectional area of the catheter is used for electrical connections to the acoustic array. In addition, the cable for transferring rotational energy may limit flexibility.

### BRIEF SUMMARY

By way of introduction, the preferred embodiments described below include methods, systems, and wobbler drive mechanisms for mechanically scanning an ultrasound transducer array. A fluid drive moves the transducer array. A pump causes fluid flow. The fluid flow transfers energy to the transducer array for moving the transducer array.

In a first aspect, an ultrasound transducer system is provided for mechanical scanning. A fluid channel has fluid operable to move a moveable transducer array.

In a second aspect, a method is provided for mechanically scanning an ultrasound transducer array. The ultrasound transducer array is driven with a fluid. The ultrasound transducer array moves in response to the driving.

In a third aspect, an ultrasound transducer system is provided for mechanical scanning. A transducer array is rotatable about an axis spaced from an emitting face of the transducer array. Alternatively, the mechanical scanning can be translational along a designated path. Combinations of rotational and translational motion are also possible. A fluid is operable to rotate and/or translate the transducer array in response to a pump.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a graphical representation of an ultrasound transducer system with a fluid drive;

FIG. 2 is a graphical representation of a fluid pump;

FIG. 3 is a graphical representation of the fluid pump of FIG. 2 in a different position;

FIGS. 4-7 are graphical representations of different drives responsive to fluid flow; and

FIG. 8 is flow chart diagram of one embodiment of a method for mechanically driving a transducer array with fluid.

### DETAILED DESCRIPTION OF THE DRAWINGS AND PRESENTLY PREFERRED EMBODIMENTS

A fluid-driven system actuates an ultrasound array for volume imaging. The pressure or flow of a fluid powers movement of the ultrasound array. Using an extended fluid channel, a wobbler may be used in probes constrained by space, such as intravascular catheters, or in probes susceptible to electromagnetic interference from electric motor-driven systems. The fluid-driven system allows the freedom to locate the fluid pump away from the ultrasonic array, reducing electrical interference.

For a cardiac or intravascular catheter implementation, the fluid may be circulated through the flexible catheter. The fluid pump may be located away from the acoustic array and processing electronics, allowing for better shielding or even amplification of the ultrasound signals before passing near the fluid pump. This location could reduce the cost of catheter-based ultrasound since no motor or pump is required in the disposable or limited-use part, such as the catheter. The power to actuate the ultrasonic array at the end of the cable or catheter is provided by the moving fluid through the catheter. The fluid channel(s) are simple, limiting the need for lengthy mechanical drive shafts. The volume for the fluid drive might be smaller than a comparable mechanical drive shaft. The fluid filled catheter or cable may remain flexible.

FIG. 1 shows an ultrasound transducer system 10 for mechanical scanning. The system 10 includes a pump 12, a fluid channel 14 with fluid and a transducer array 16. Additional, different or fewer components may be provided. For example, the system 10 also includes a catheter housing and an ultrasound imaging system.

The pump 12 is a fluid pump, such as a suction or reciprocating pump. The fluid pump provides constant or variable pressure or flow in one direction or reciprocating between two directions. Fluid pumps are used in many medical systems and are capable of imparting large linear forces of many pounds or more. As an example, syringe pumps (e.g., the Harvard PHD2000 syringe pump) may impart 15 PSI to 1000 PSI with flow rates ranging from 0.003 μl/min to 220 ml/min. Higher or lower pressures or flow rates may be provided. The pressure used is sufficient to move the transducer array 16 but avoid causing leaks in the fluid channel 14.

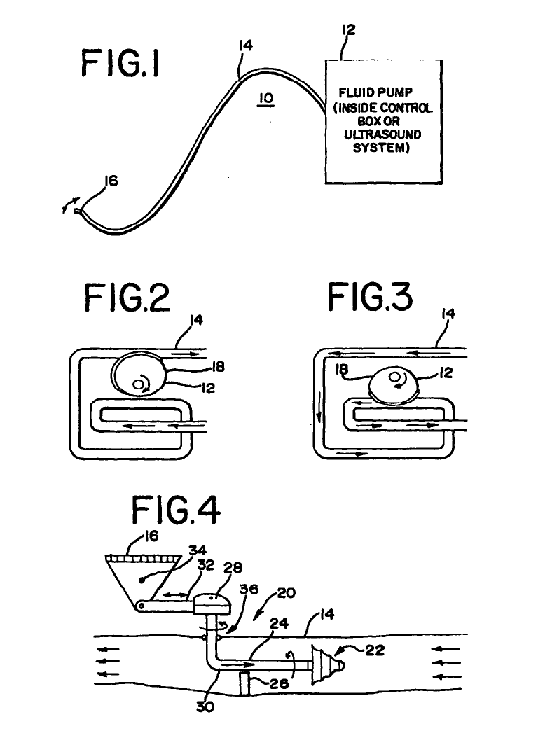

FIGS. 2 and 3 show an embodiment of a reciprocating pump 12. The pump 12 is an offset structure 18, such as an off-center mounted wheel, oblong device, or other structure. An axis on the off-set structure 18 connects directly or through gearing or belts to an electric motor. As the off-set structure 18 rotates, the off-set structure 18 contacts different portions of the flexible fluid channel 14 in different directions. Pressure is applied and released from the fluid channel 14, causing reciprocating flow of fluid in the channel 14. The off-set structure 18 is rotated in one direction or rotates back and forth in two different directions.

The pump 12 connects with the fluid channel 14. For example, FIGS. 2 and 3 show the pump 12 connecting with the fluid channel 14 through periodic contact. As another example, the pump 12 has impellers, paddles, turbines, propellers or other mechanisms for creating fluid flow through contact. The fluid channel 14 terminates at, is fastened to or passed adjacent to the pump 12.

The pump 12 is positioned to remain outside of a patient while the transducer array 16 is positioned to be within the patient. The pump 12 is within a probe control box or housing, within an ultrasound imaging system or within a separate pump housing. The fluid channel 14 interconnects the pump 12 with the transducer array 16. For example, the pump 12 drives fluid through the fluid channel 14 in a catheter, and the fluid motion is converted into mechanical movement of the ultrasonic array 12 by a mechanical fixture or drive located at the transducer array 16. In alternative embodiments, the pump 12 is positioned adjacent to the transducer array 16, such as being positioned in a hand-held transducer array housing of a probe used externally to the patient.

The fluid channel 14 is a tube, a cavity, a container, a chamber, a reservoir, or combinations thereof. The fluid channel 14 is plastic, rubber, ceramic, wood, metal, semiconductor, PVC, combinations thereof or other now known or later developed materials. The fluid channel 14 is entirely a same material or structure or includes different materials or structures at different locations. For example, the fluid channel 14 extends through a plastic pump 12, along a rubber hose, and into a metal transducer array housing. The fluid channel 14 is generally straight, but may include bends, elbows or other curves. The fluid channel 14 is flexible, but may have rigid portions or be entirely rigid.

The fluid channel 14 extends from the pump 12 to adjacent to the transducer array 16 in one or more path ways, such as a single channel or a loop. In alternative embodiments, the fluid channel 14 extends part way from the pump 12 to the transducer array 16, and fluid flow or pressure is converted to mechanical rotation and/or translational motion for transferring energy to the transducer array 16.

The fluid channel 14 contains fluid. The fluid is a gas or liquid, such as water or saline. The fluid channel 14 is sealed to prevent leakage of the fluid. Alternatively, the fluid channel 14 is unsealed. A relief valve may be provided, such as adjacent the pump 12.

The fluid moves the transducer array 16. For example, flow or pressure from the fluid within the fluid channel 14 is converted to mechanical energy, causing the transducer array 16 to rotate or translate. The pump 12 causes the fluid to move the transducer array 16.

The fluid moves within the fluid channel 14 in a substantially continuous flow. The flow or pressure of the fluid is maintained substantially constant during mechanical scanning. By providing a looping fluid channel 14 (e.g., a source path and a return path), the fluid flows in one direction through the loop. The moving fluid also removes some of the heat dissipated from the array 16. To maintain an about constant wobble or transducer movement rate, the flow or pressure is kept substantially constant. The flow or pressure decreases when stopping the mechanical scan and increases when starting the mechanical scan, but provides substantially continuous flow after starting and before stopping. In alternative embodiments, the flow or pressure is varied, such as increasing or decreasing a flow rate as a function of transducer location. For example, the transducer array 16 is moved more quickly or slowly at edge regions or the ends of the mechanical scan. As another example, the speed of the wobbling ultrasonic transducer array 16 is varied by the rate of fluid flow or different gear ratios in the mechanical conversion. The flow rate may vary to account for changes in rate from the mechanical conversion, providing substantially constant motion of the transducer array 16.

In another embodiment, the fluid moves within the fluid channel 14 in a substantially reciprocating flow. The fluid moves back and forth or periodically changes direction of pressure or flow in the fluid channel 14. The fluid channel 14 is a single path or a looping path between the pump 12 and the transducer array 16. Depending on the frequency, fluid type, fluid mass, length of the fluid channel 14, and strength of the fluid channel 14, reciprocating motion moves the transducer array 16. The pressure of the fluid is varied at the desired frequency of mechanical movement. Depending on the frequency of operation and characteristics of the fluid and fluid channel 14, the transducer array 16 is actuated by small fluid displacements under high pressure or with larger movements under low pressure.

Referring to FIGS. 4-7, the fluid flow or pressure is converted to mechanical movement by a drive 20. The drive 20 generates constant or reciprocating mechanical force for application to the transducer array 16. The mechanical force is rotational or translational. The drive 20 uses gears, belts, pulleys, pistons, cams, bearings, axles, universal joints, seals, wires, conductors, metal brushes, combinations thereof, or other now known or later developed mechanism for providing mechanical force from the fluid force.

FIG. 4 shows a drive 20 with a turbine 22, axle 24, support 26, cam 28, universal joint 30, belt 32 and axis 34. Additional, different or fewer components may be provided, such as providing additional supports for one or more of the components. The turbine 22 is within the fluid channel 14. A propeller or other structure for causing rotation from flow of fluid may alternatively be used. The rotation of the turbine 22 causes the axle 24 to rotate. The support 26 includes one or more bearings or a seat for holding the axle in place. The universal joint 30 transfers the rotation of the axle 24 along another axis. In alternative embodiments, the axle 24 has no or additional universal joints, bends, elbows or other changes in direction. The axle 24 extends out of the fluid channel 14. A seal 36 prevents fluid from leaking. The rotation of the axle 24 rotates the cam 28. The cam 28 converts the rotation motion into a linear motion. For example, the belt 32 or another linkage connects the cam 28 to the transducer array 16. The axle 24 mounts to the cam 28 off-center, causing the linear motion of the transducer array 16 about the axis 34. As the cam 28 rotates, the transducer array 16 wobbles about the axis 34. Other turbine 22 or propeller structures may be used. Alternatively, in the absence of axis 34, purely translational array motion is possible along a guided pathway. In another embodiment, a worm-drive could convert rotational motion of the axle to translational motion of the array.

FIG. 5 shows another embodiment of the drive 20. The drive 20 includes paddles 42 connected about an axis 40 with a bulge 44 of the fluid channel 14. The paddles 42 form a turbine, propeller or paddle wheel. The paddles 42 rotate about the axis 40. A bearing may be provided. The transducer array 16 connects with one of the paddles 42 or another structure connected with the paddles 42. For example, gearing may be used. The transducer array 16 is oriented as a one dimensional array parallel but spaced from the axis 40, in a plane of rotation or other orientation. Continuous fluid flow in the fluid channel 14 applies force to some of the paddles 42. The bulge 44 exposes the paddles 42 on one side of the axis 40 to more flow than another side, causing rotation of the paddles 42 in response to continuous or reciprocating flow. As the paddles 42 move, the transducer array 16 is moved. As the transducer array 16 rotates, a 360 degree or lesser angle mechanical sweep or scan is performed. The fluid channel 14 is acoustically matched to water, blood, tissue or the fluid. A fluid tight seal may be avoided by providing the drive 20 within the fluid channel 14. Electrical contacts with the transducer array 16 are provided with tabs or brushes contacting a fixed ring of electrodes or connectors. Other rotational to fixed electrical connectors may be used.

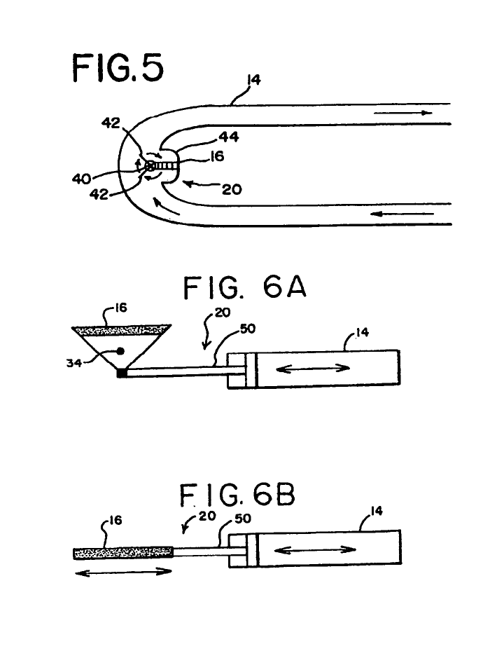

FIG. 6A shows yet another embodiment of the drive 20. The drive 20 includes a piston 50 extending from the fluid channel to the transducer array 16. The fluid pressure at the end of the fluid channel 14 moves the piston 50. Reciprocating fluid pressure moves the piston 50 in a back and forth motion. The transducer array 16 is rotatably mounted about the axis 34 by a lever. The piston 50 moves the transducer array 16 about the axis 34, wobbling the transducer array 16.

FIG. 6B shows a variation of the embodiment of FIG. 6A. In this embodiment, the piston 50 is driven by fluid pressure as in FIG. 6A, but the piston causes a linear translation of the transducer array 16. The transducer array 16 may be oriented in line with the axis of translation to provide an extended field of view or may be oriented transversely to the axis of translation to provide a volumetric image.

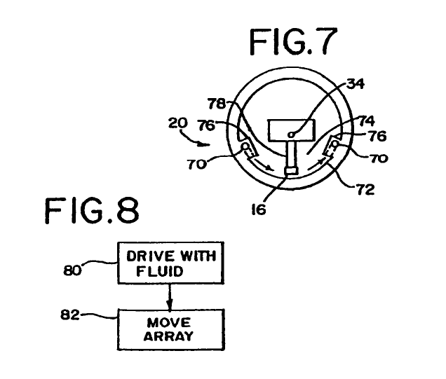

FIG. 7 shows another embodiment of the drive 20. The drive 20 includes one or more ports 70 in a housing 72 of a fluid chamber 74. The ports 70 connect with the fluid channel 14. As fluid flows through the ports 70, the fluid in the chamber 74 also flows. By angling one or more ports 70 or by providing entrance and exit ports 70, the fluid in the chamber 74 is caused to flow in a desired direction. By reversing the fluid flow, the fluid in the chamber 74 flows in a different direction. The transducer array 16 and/or a paddle 78 extend within the chamber 74 and rotate about the axis 34. The paddle 78 and transducer array 16 substantially block the flow, or gaps are provided. The flowing fluid in the chamber 74 causes the transducer array 16 or paddle 78 to move with the flow, such as towards or away from one or more ports 70. The stops 76 or flow characteristics limit the movement of the transducer array 16.

Other drives may be used to convert fluid motion to mechanical motion. Combinations of different drives may be used.

Referring to FIGS. 1 and 4-7, the transducer array 16 is a 1D, 1.25D, 1.5D, 1.75D, 2D, multidimensional or other now known or later used array of transducer elements. Piezoelectric or micro machined (e.g., CMUT) elements may be used. The transducer array 16 is linear or curved. In an alternative embodiment, a single transducer element is provided.

The transducer array 16 is moveable. For example, the transducer array 16 is mounted adjacent an axis to rotate about the axis. As another example, the transducer array 16 is mounted on a lever arm (e.g. a block, housing or other structure) to rotate about an axis spaced away from the transducer array 16. As yet another example, the transducer array 16 includes guides, bearings, sliders or other structures to translate with or without rotation. Any now known or later developed wobbler array structures may be used.

The transducer array 16 is allowed to rotate or move without limit. Alternatively, the transducer array 16 is limited by the drive 20 or stops 76. For example, pegs, plates, blocks or other structures prevent movement of the transducer array 16 past a particular position. One or more stops are provided to limit movement in one direction or to a range of motion. For example, the fluid moves the moveable transducer array 16 between opposing stops 76 in response to different flow directions.

An encoder, sensor or networks of sensors may provide position feedback to the fluid pump 12 and the ultrasound system. For example, a rotary or linear encoder for digital feedback, a rotary or slide potentiometer for analog feedback, an optical sensor, a capacitive sensor, or any other currently known or later developed sensor indicates position for image reconstruction.

The system 10 or part of the system 10 is provided within a probe housing. The transducer array 16 is within or on the probe housing. Any probe housing may be used, such as a hand held probe. Probes operable to ultrasonically scan from within a patient may be used, such as a catheter, intraoperative probe, transesophageal probe, or endocavity probe.

FIG. 8 shows a method for mechanically scanning an ultrasound transducer array. Additional, different or fewer acts may be provided. The acts are performed in the order shown or a different order. The method is implemented with the system 10 or drives 20 of FIGS. 1-7 or different systems or drives.

In act 80, the ultrasound transducer array is driven with a fluid. Driven includes applying force with fluid, such as through flow and/or pressure. Fluid in a fluid channel is pumped. Substantially continuous flow of the fluid in a first direction is generated, or substantially reciprocating flow of the fluid in at least two directions is generated. The reciprocating flow may be in a single fluid path or through a fluid loop. The fluid path may be short, such as associated with a hand held probe, or may extend 10 or more centimeters, such as associated with a tube within a catheter, intraoperative probe, transesophageal probe, or endocavity probe. The fluid flow or pressure rotates a turbine, rotates a paddle, moves a piston, combinations thereof or operates another drive for converting to mechanical motion.

In act 82, the ultrasound transducer array moves in response to the driving. The movement is back and forth, such as wobbling, or continuous. Rotational, translation or other motion is generated.

Referring again to FIG. 1, the pump 12 provides sufficient pressure to the fluid given the characteristics of the fluid channel 14 and the drive 20. The pressure drop in the fluid channel 14 is considered. The pressure drop depends on the flow rate, which depends on the power to be delivered. To wobble a hypothetical array mounted a on a solid cylinder with a radius of 3 mm, length of 10 mm, with a mass density that is the same as water, ρ=1000 kg/m³, the moment of inertia (1/2*mass*radius²) is 1.3e−9 kg-m². Assuming enough torque to accelerate the mass from rest to a full revolution for each frame at a frame-rate of 20 Hz, the transducer completes a full rotation every 0.05 s. This corresponds to an constant angular acceleration of α=2*2π/t²=5 krad/s². The constant torque for this acceleration is 6.4e−6 N-m. The energy to apply this torque over one full 360-degree rotation (2*pi) is about 4e−5 J. The average power required for the specified movement of this hypothetical wobbler is about 0.8 mW, but any of the characteristics in the hypothetical may be different. This power estimate does not include frictional forces, but does assume that the transducer motion starts from rest at the beginning of each frame or revolution. Embodiments in which the actual transducer revolves continuously may require lower average power levels, only requiring enough power to compensate for frictional losses.

Since the power transfer from the fluid to the hypothetical transducer array may be inefficient, about 10 mW of power flow in the fluid is used as an order of magnitude estimate to determine the necessary fluid pressure and flow rates. A flexible Viton microtube with inner diameter of D=1.6 mm (cross-sectional area A=2e−6 m²) may be capable of withstanding about 15 psi (about 1e5 Pa) of pressure continuously. For a power flow of 10 mW at a pressure of 1e5 Pa, the average flow velocity (V) is about 0.05 m/s since the power flow is the product of the pressure, cross-sectional area, and average fluid velocity across the cross-section. This corresponds to a volume flow rate of 1e−7 m³, or about 6 mL/min, which is a typical flow rate for fluid syringe pumps used in medical applications. If the fluid is water (ρ=1000 kg/m³, μ (dyn. viscosity)=8.9e−4 Pa·s), the Reynolds number (Re) for this tube diameter and velocity is Re=ρ*V*D/μ=90, which corresponds to laminar flow. For laminar flow, the pressure drop over L=1 meter of horizontal tubing is calculated as Δp=32*L*u*V/D²=553 Pa, which is roughly half a percent of the input pressure. In general, the pressure drop and corresponding energy loss may be ignored at these low flow rates. In case the tubing is oriented vertically so that the tube input is 1 meter below the transducer, then the pressure drop increases to about 1e4 Pa, or about 10% of the input pressure. The effects of bends and fittings are not included in the calculations.

The 1.6-mm inner-diameter tube may be small enough for transesophageal probe applications, but may be too large for catheter applications. For the same power delivery of 10 mW and a same 15 PSI of input pressure, the velocity of the flow increases as the tube diameter shrinks. Although the flow remains laminar, the pressure drop becomes a significant fraction of the input pressure:

| | Avg. Velocity | | Press. Drop | Drop as % |
|---|---|---|---|---|
| Tube I.D (mm) | (m/s) | Reynolds # | (Pa) | input |
| 1.6 | 0.05 | 90 | 553 | 0.553 |
| 1 | 0.127 | 143 | 3600 | 3.6 |
| 0.75 | 0.226 | 190 | 11460 | 11.46 |
| 0.5 | 0.5 | 286 | 58000 | 58 |

At 0.5 mm, more than half of the power or energy is lost in transport through the fluid channel. However, the amount of power necessary to wobble the transducer array may also shrink as the array and tube size shrink. For enough flow for 5 mW with the same input pressure, the pressure drop is only half as large. The pressure drops scale linearly with tube length, so a 2-m tube may have twice the pressure drop and energy/power loss.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. An ultrasound transducer system for mechanical scanning, the system comprising:
a moveable transducer array; and
a fluid channel with fluid operable to move the moveable transducer array.

2. The ultrasound transducer system of claim 1 wherein the moveable transducer array comprises a wobbler array.

3. The ultrasound transducer system of claim 1 wherein the moveable transducer array is within a catheter, intraoperative probe, transesophageal probe, or endocavity probe.

4. The ultrasound transducer system of claim 1 wherein the fluid channel comprises a tube and the fluid comprises a gas or liquid;
further comprising:
a pump connected with the fluid channel.

5. The ultrasound transducer system of claim 1 wherein the moveable transducer array comprises a one dimensional array of elements rotatable about an axis spaced away from the one dimensional array of elements.

6. The ultrasound transducer system of claim 1 wherein the fluid is operable to move the moveable transducer array in response to substantially continuous flow of the fluid in the fluid channel.

7. The ultrasound transducer system of claim 1 wherein the fluid is operable to move the moveable transducer array in response to substantially reciprocating flow of the fluid in the fluid channel.

8. The ultrasound transducer system of claim 1 further comprising:
a turbine within the fluid channel;
an axle operatively connecting the turbine with the moveable transducer array.

9. The ultrasound transducer system of claim 1 further comprising:
a plurality of paddles within the fluid channel, the plurality of paddles operatively connected with the moveable transducer array.

10. The ultrasound transducer system of claim 1 further comprising:
a piston connected with the fluid channel and the moveable transducer array.

11. The ultrasound transducer system of claim 1 further comprising:
a plurality of stops adjacent the moveable transducer array;
wherein the fluid is operable to move the moveable transducer array between the stops in response to different flow directions.

12. The ultrasound transducer system of claim 1 wherein the moveable transducer array comprises a one dimensional array of elements operable to translate.

13. The ultrasound transducer system of claim 1 further comprising:
a sensor operable to determine a location of the moveable transducer array.

14. A method for mechanically scanning an ultrasound transducer array, the method comprising:
driving the ultrasound transducer array with a fluid; and
moving the ultrasound transducer array in response to the driving.

15. The method of claim 14 wherein moving comprises wobbling.

16. The method of claim 14 wherein moving comprises rotating.

17. The method of claim 14 wherein driving comprises moving the fluid in a tube within a catheter, intraoperative probe, transesophageal probe, or endocavity probe.

18. The method of claim 14 wherein driving comprises pumping the fluid in a fluid channel.

19. The method of claim 14 wherein driving comprises generating substantially continuous flow of the fluid in a first direction.

20. The method of claim 14 wherein driving comprises generating substantially reciprocating flow of the fluid in at least two directions.

21. The method of claim 14 wherein driving comprises rotating a turbine, rotating a paddle, moving a piston, or combinations thereof with the fluid.

22. The method of claim 14 wherein moving comprises translating the transducer array.

23. The method of claim 14 wherein moving comprises combinations of translation and rotation.

24. An ultrasound transducer system for mechanical scanning, the system comprising:
a transducer array rotatable about an axis spaced from an emitting face of the transducer array;
a pump;
a fluid operable to rotate the transducer array in response to the pump.

25. The ultrasound transducer system of claim 24 wherein the transducer array is within a catheter, intraoperative probe, transesophageal probe, or endocavity probe, and wherein the pump is positioned to remain outside of a patient while the transducer array is positioned to be within the patient;
further comprising a fluid channel extending from the pump and containing the fluid.

26. The ultrasound transducer system of claim 24 further comprising:
a turbine, a paddle, a piston or combinations thereof responsive to flow of the fluid.
